(19) United States
(12) Patent Application Publication
(10) Pub. No.: US 2004/0230182 A1
(43) Pub. Date: Nov. 18, 2004

(54) Drug delivery through encapsulation

(75) Inventors: Kenneth T. Heruth, Edina, MN (US); Mark S. Lent, Brooklyn Park, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/745,720

(22) Filed: Dec. 23, 2003

Related U.S. Application Data
(60) Provisional application No. 60/436,841, filed on Dec. 27, 2002.

(51) Int. Cl. A61K 9/22

(52) U.S. Cl. 604/891.1

(57) ABSTRACT

An implantable drug delivery device having a drug releaser that releases a drug from a capsule in the catheter. The drug delivery device includes a reservoir configured to store a drug capsule, a catheter, a pump configured to move the drug capsules into the catheter, and a drug releaser. The drug releaser is connected to the catheter for freeing at least a portion of the drug from one or more of the drug capsules while in the catheter.

### BACKGROUND OF THE INVENTION

1. Field of the Invention

This invention relates to drug delivery techniques, and more particularly relates to such techniques for treating neurodegenerative disorders.

2. Description of Related Art

There are a number of conventional apparatuses and methods for drug delivery to a patient. Implanted drug delivery systems have involved two general approaches. One approach is to use an implanted drug administration device, wherein drugs are pumped from a reservoir to a target site within a patient. See e.g., U.S. Pat. Nos. 5,711,316, 4,692,147, 5,462,525, and 4,003,379. The reservoir can be replenished as necessary through a replenishing port, and without removal of the implanted device from the patient. Some drugs are not stable when dissolved in a vehicle delivery solvent. Other drugs are stable for only a short period of time when dissolved in a solvent. Some drugs are stable for example for only 30 to 90 days. After that time, the drug will precipitate out of solution, or the drug molecule may be altered. When a significant amount of the drug has degraded, the solution has to be replaced, even if a useful quantity is still available in the reservoir. When this occurs, the patient must visit a medical center to have the reservoir emptied of the degraded solution and refilled with non-degraded solution.

Most conventional devices store the drug to be delivered in a reservoir, with the drug dissolved in a liquid solvent, such as water or saline. The stored solution is quite dilute, e.g. 1-5% of the drug compared to 95-99% carrier. Further, the reservoir in the device for the delivered drug must be large enough for the requisite solvent, and the reservoir must be replenished frequently. Thus, there is a need for devices and methods that can deliver drugs that are not stable when dissolved in a solvent, and to do so in a controlled manner. There is also a need for smaller devices that do not have the large reservoir required by conventional devices and methods.

A second approach has been to use implanted capsules that will permit the drug within the capsule to transfer outside of the capsule wall by diffusion and/or by the dissolving of the capsule wall. See e.g., U.S. Pat. Nos. 5,106,627 and 5,639,275. A major drawback with this approach is that it is a passive drug delivery system; drug delivery rate cannot be controlled after implantation of the capsule within the patient. Further, additional capsules must be implanted after earlier capsules are dissolved or spent.

U.S. Pat. No. 6,458,118, which is incorporated herein by reference, describes a system in which small amounts of the drug are encapsulated in a biodegradable polymer. The encapsulated particles and a carrier fluid are stored in the reservoir of an implantable drug delivery device. The drug is freed from the polymer in the drug device reservoir and dissolves in the carrier fluid and is then delivered by a pump through a catheter to the desired location in the body. While this system overcomes the problems described above, there are some situations where it is desirable to not free the drug until just before it will be infused. As one example, sometimes it is desirable to infuse very small amounts of liquid, e.g. less than 50 microliters per day. The fluid volume in an electromechanical pump and a catheter will typically be several milliliters. The drug can take several days to be infused into the body after it is released from the polymer capsule. As another example, in some situations the pump will normally be turned off. It will be started and infuse drug only in response to a physiological event, e.g. an epileptic seizure. The drug may remain in residence in the implanted pump system for periods lasting several months. In these situations, it is more desirable to free the drug from the polymer encapsulation closer to the site of infusion into the body.

The present invention is directed to these particular difficulties which the prior art fails to address.

### SUMMARY

A preferred form of the invention can provide controlled drug delivery. The drug is stored within an implantable device in encapsulated form. Small amounts of the drug, e.g. 1 microgram, are encapsulated in an inert material, e.g. a stable polymer. The encapsulated drug is stored in a reservoir of the implantable device. Further, there may be a supply of pure carrier in the implantable infusion device. This can be a separate carrier, such as water, stored in a separate reservoir system. In addition, the supply of pure carrier can be replenished.

The carrier can also be a body fluid, such as cerebrospinal fluid from the patient's body. This concept of dissolving a drug into a stream of recirculating body fluid is disclosed in U.S. Pat. No. 5,643,207, which is incorporated herein by reference.

When drug infusion is desired, some of the encapsulated drug is metered by the implantable device into the carrier fluid. The capsules are smaller than any dimension of the pump or interconnecting passages, and will be moved by the pump with the carrier fluid into the catheter. The drug is freed from the polymer capsules in the catheter, close to the site of infusion into the body. The drug will dissolve in the carrier fluid and be carried into the body.

The capsules can be broken in any suitable manner by a drug releaser involving any suitable mechanism, including: ultrasonic waves, mechanical crushing or grinding; chemically dissolving or splitting; applying an electrical current to potentiate a chemical reaction; heating; or applying pressure (e.g. hydrostatic pressure). Thus, in accordance with the present invention, the drug releaser is any device, chemical or other mechanism that releases drug from a capsule, including, but not limited to, an ultrasonic sound emitter, a mechanical crushing or grinding device, a chemical dissolving or chemical splitting apparatus, an electrical current emitter, a heater, or a pressure device.

### BRIEF DESCRIPTION OF THE DRAWINGS

FIGS. 1A and 1B are diagrammatic illustrations of the present invention implanted in a patient.

FIG. 2 is a diagrammatic illustration of a preferred embodiment of the present invention, including an electromechanical pump, encapsulated drug in a reservoir, and a catheter.

FIG. 3 is a diagrammatic illustration of another preferred embodiment of the implantable drug delivery device of the present invention, including an electromechanical pump, reservoir, encapsulated drug in a premixing vessel, and a catheter.

FIG. 4 is a block diagram of an exemplary embodiment dual lumen catheter and supporting pumps and reservoirs.

FIG. 5 is a block diagram of another exemplary embodiment dual lumen catheter and supporting pumps, valves and reservoirs.

FIG. 6 is a block diagram of another exemplary embodiment dual lumen catheter and supporting pumps, interlumen valve and reservoirs.

### DESCRIPTION OF THE PREFERRED EMBODIMENTS

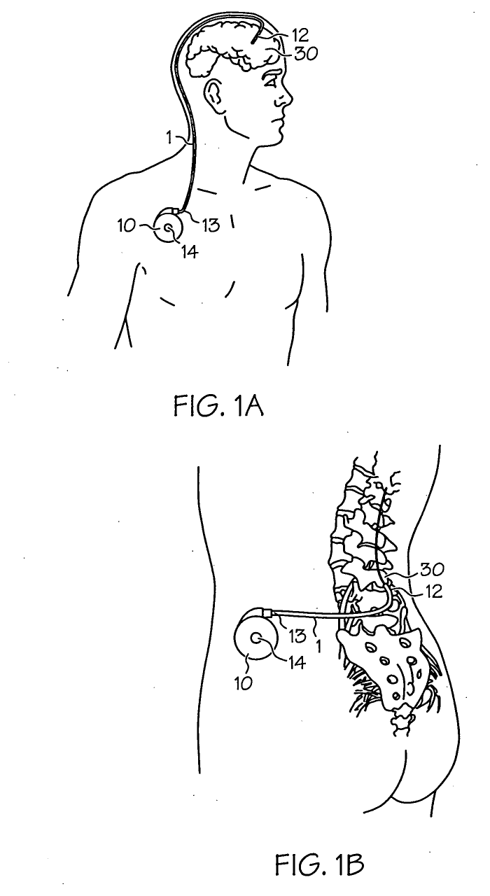

Referring to FIGS. 1A and 1B, an implantable drug delivery system or device 10 made in accordance with the preferred embodiment may be implanted below the skin of a patient. The implantable device 10 has a port 14 into which a hypodermic needle can be inserted through the skin to inject a quantity of capsules 31 containing a medication or drug 29. Catheter 1 is positioned to deliver the agent to specific target sites 30 in a patient.

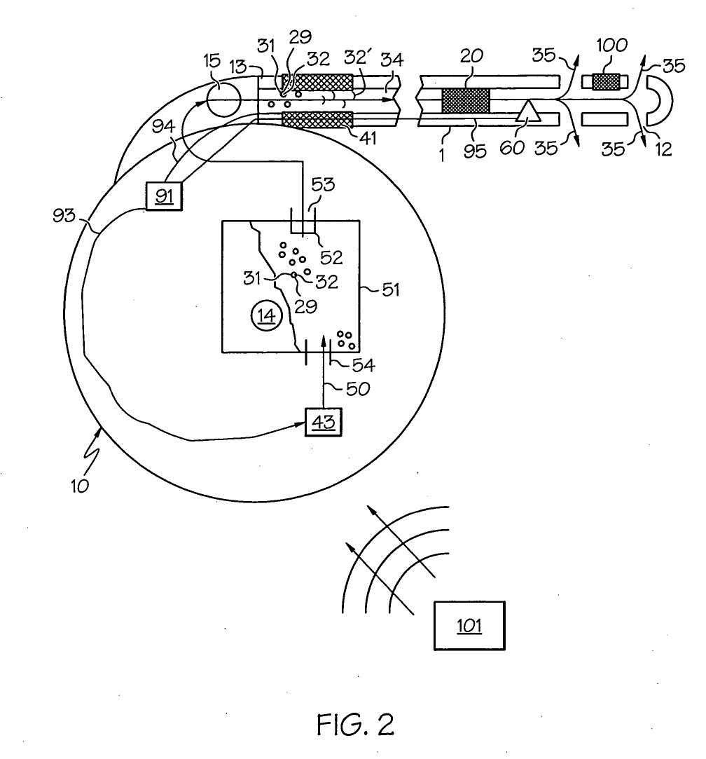

As further shown in FIG. 2, drug 29 is maintained in capsule 31 by capsule wall 32. The term “drug” is used in this application to mean any therapeutic agent including pharmaceuticals and bioactive substances such as a cell, a protein, or a genetic substance. The term “capsule” is used in this application to mean both physical containers for holding or storing a drug, as well as microencapsulated drugs, microemulsion and mycells. A capsule must be of small enough size to fit within the reservoir and within the catheter. A capsule does not necessarily need to be soluble as long as there is a mechanism for releasing the drug from the capsule (such as by breaking or splitting the capsule). An exemplary capsule may be made of a polymer wall 32. Microencapsulated drugs, microemulsions and mycells are well known in the art and the mechanism, molecules or substance for containing or surrounding the drug in a microencapsulated drug, microemulsion or mycell is considered to be a capsule. The term “drug capsule” is used in this application to mean the combination of the drug in the capsule.

As shown in FIG. 2, carrier fluid 50 is supplied to reservoir 51 through entryway 54. Carrier fluid 50 can be any suitable fluid, including bodily fluids.

A pump moves the capsules 31 from the reservoir to the catheter. The term pump is used in this application to mean any device capable of moving the capsules 31 from the reservoir to the catheter including, but not limited to, electrochemical pumps, and electromechanical pumps such as peristaltic, solenoid and piezoelectric pumps. In one embodiment, electromechanical pump 60 pumps the mixture of capsules 31 and fluid 50 to catheter 1. The capsules will be smaller than any passages in the electromechanical pump.

For example, the peristaltic pump disclosed in US Patent No. 4,576,556 has a tube with an inside diameter of 0.5 mm. The capsules may have a diameter smaller than 0.01 mm, and will pass directly through the pump tube.

Inside catheter 1, the polymer capsules can be broken by a drug releaser, freeing the drug 29 from the capsule 31. The drug 29 will then dissolve in the carrier fluid 50. Catheter 1 then conveys the mixture through proximal end 13 and lumen 34 of catheter 1, and through openings 35 at distal end 12 of catheter 1, to the target site 30 within the patient.

The capsules can be broken in any suitable manner by a drug releaser involving any suitable mechanism, including: ultrasonic waves, mechanical crushing or grinding; chemically dissolving or splitting; applying an electrical current to potentiate a chemical reaction; heating; or applying pressure (e.g. hydrostatic pressure). Thus, in accordance with the present invention, the drug releaser is any device, chemical or other mechanism that releases drug from a capsule, including, but not limited to, an ultrasonic sound emitter (such as ultrasonic sound emitter 41), a mechanical crushing or grinding device, a chemical dissolving or chemical splitting apparatus, an electrical current emitter, a heater, or a pressure device. In the case of microencapsulated drugs, one exemplary drug releaser is an acidic chemical such as citric acid that releases the drug from microencapsulation upon exposure to the citric acid.

The present invention includes both open loop (sometimes referred to as non-responsive) therapy as well as closed loop (responsive) therapy.

In the case of closed loop therapy, device 10 is capable of changing the delivery of drug 29 based on reading from a sensor 100 measuring conditions at a target site 30 within the patient. Sensor 100 could for example sense pressure, temperature, an electrical signal such as ECG or EEG, motion, or concentration of a substance in an organ or body fluid, e.g. oxygen, carbon dioxide, or a protein.

Alternatively, device 10 can be programmed for drug delivery and/or drug delivery by device 10 can be changed from outside the patient via a telemetry unit 101. By way of example, as shown in FIG. 2, device 10 can have an electrical control circuit 91 which controls ultrasonic sound emitter 41 via sound emitter control pathway 95 and the ultrasonic sound waves 40 therefrom. Those skilled in the art will recognize that electrical control circuit 91 can also control the flow of carrier fluid 50 to reservoir 51 via control carrier fluid pathway 93 and controlling carrier fluid metering device 43. Those skilled in the art will also recognize that electrical control circuit 91 can also control pump 60 via pump control pathway 94. Thus, electrical control circuit 91 can be used to control the pumping of the mixture of dissolved drug 29 and carrier fluid 50 to patient site 30 as desired.

One embodiment is contemplated such that the above device and method for drug delivery will be able to permit drug delivery for about a one-year period. In this embodiment, enough encapsulated drug would be stored in device 10 and last for the expected time period. At the end of that time period the implantable device 10 can be replenished via port 14 or explanted as desired. In another embodiment, the carrier fluid and drug can be replenished through a refill port 14 by using a hypodermic needle and syringe to access the reservoir. In another embodiment, a filter 20 can be placed in the catheter downstream from the drug releaser mechanism. The filter will have a pore size such that the carrier fluid and dissolved drug will pass through to the outlet ports 35. The empty, broken capsules are trapped by the filter in the inlet area of the catheter. The broken capsules may also be removed periodically from the catheter via a catheter access port 15. A hypodermic needle and syringe can be used to access the catheter through the catheter access port.

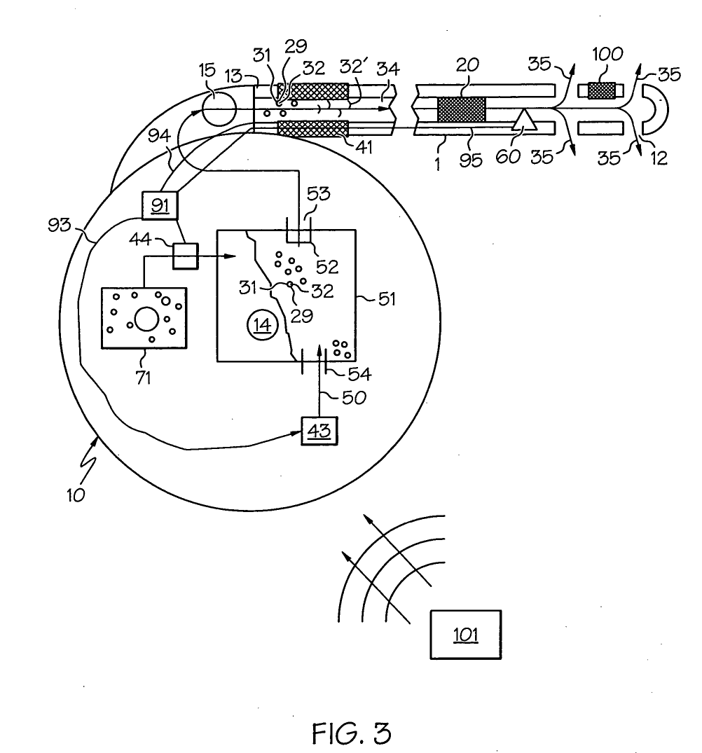

As shown in FIG. 3, in another embodiment, encapsulated drug 29 may be stored in a premixing vessel 71, and outside of reservoir 51. Drug 29 can be metered from premixing vessel 71 into reservoir 51 as needed via any suitable metering device 44. If more accurate drug infusion is required, a drug concentration sensor 90 can be placed in catheter lumen 34. Sensor 90 can send sensor signals via signal pathway 92 to an electrical control circuit 91 in device 10. The control circuit 91 controls drug metering device 44 via drug control signal pathway 45 so that drug metering device 44 only meters drug 29 into the reservoir 51 when the concentration of the drug 29 within reservoir 51 falls to a preset limit. The sensor 90 can also measure the concentration of drug 29 and electrical circuit 91 can control fluid metering device 43 via fluid control signal pathway 93 to precisely infuse into reservoir 51 the amount of carrier fluid 50 that is required to deliver a specified amount of drug 29 to the patient. In FIG. 3, encapsulated drug 29 can be provided to premixing vessel 71 through port 15.

By using the foregoing techniques, numerous drug delivery applications can be achieved to treat numerous conditions, including motor disorders, with a controlled degree of accuracy previously unattainable.

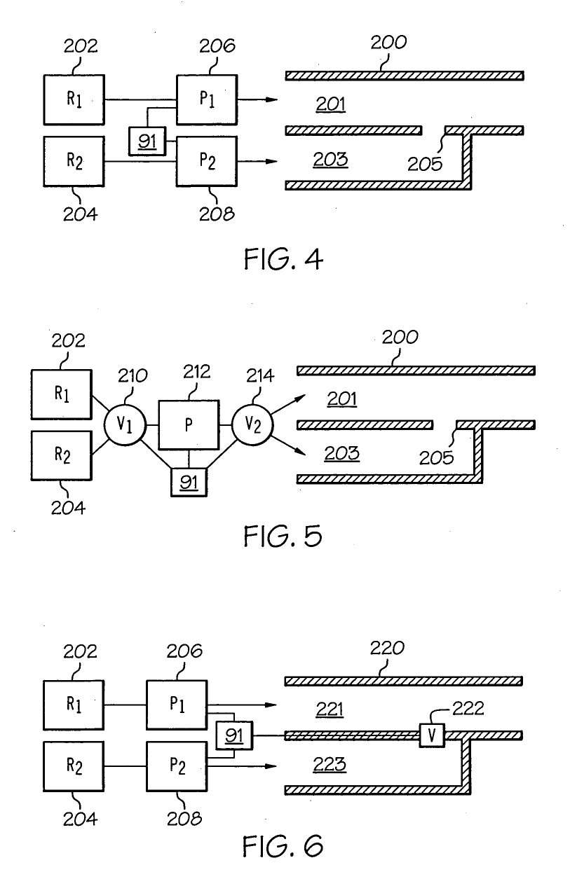

Those skilled in the art will also recognize that drug delivery in accordance with the present invention can be achieved by measuring the physiological conditions at the patient target site 30. For example, the measurement of hyperexcited cells can be detected with a sensor 100 as shown in FIGS. 2 and 3, or sensor 403 as shown in FIG. 4. Further, sensor 100 can send a signal to electrical control circuit 91, which as shown in FIG. 3 as an example, controls the mixing of drug 29 and carrier fluid 50. The sensor 403 shown in FIG. 4 can also be used to send a signal to an electrical control circuit 91, which in turn can regulate drug delivery from an implantable drug delivery device, including those shown in FIGS. 2 and 3.

FIGS. 4-6 illustrate various embodiments of a catheter of this invention having multiple lumens for use with a chemical type drug releaser. For example, in the example of drug capsules that are microencapsulated drugs, microemulsion or mycells, one form of a drug releaser is an second lumen 203 or 223 in the catheter 200 or 220 and movement of a chemical or other agent (such as, but not limited to, an acidic agent) through the second lumen to be mixed with the drug capsules in the first lumen 201 or 221. This mixing of a chemical or other agent with the drug capsules results in a break down of the capsule and release of the drug. The chemical passes through port 205 or through valve 222 from the second lumen 203 or 223 into the first lumen 201 or 221.

The embodiment of FIG. 4 includes two reservoirs 202 and 204 connected respectively to two pumps 206 and 208. Pump 202 moves drug capsules and a carrier fluid from reservoir 202 into lumen 201 of catheter 200. Pump 204 moves a chemical or other agent from reservoir 204 into lumen 203 and through port 205 into lumen 201 where the chemical or other agent mixes with the drug capsules resulting in release of the drug from the capsules. Control circuit 91 controls the pumps 206 and 208.

The embodiment of FIG. 5 includes two reservoirs 202 and 204 both connected to a valve 210. Valve 210 directs a fluid from one of reservoirs 202 and 204 to pump 212. Pump 212 moves the fluid selected from one of reservoirs 202 and 204 to one of lumens 201 and 203 depending on position of valve 214. Control circuit 91 controls the valves 210 and 214 as well as pump 212.

The embodiment of FIG. 6 is similar to the embodiment of FIG. 4 except that it includes a valve 222 for controlling movement of a chemical or other agent from the second lumen 223 into the first lumen 221. Valve 222 is controlled by control circuit 91.

Those skilled in the art will recognize that the capsules can be broken in any suitable manner, including: ultrasonic waves, mechanical crushing or grinding; chemically dissolving or splitting; applying an electrical current to potentiate a chemical reaction; heating; or applying pressure (e.g. hydrostatic pressure).

Those skilled in that art will recognize that the preferred embodiments may be altered or amended without departing from the true spirit and scope of the invention, as defined in the accompanying claims.

We claim:

1. An implantable drug delivery device comprising:
a reservoir configured to store a drug capsule;
a catheter having a proximal end and a distal end, wherein the catheter has a first lumen and wherein the proximal end is connected to the reservoir for receiving the drug capsule from the reservoir into the first lumen;
a pump configured to convey the drug capsules to the catheter; and
a drug releaser connected to the catheter for freeing at least a portion of a drug from one or more of the drug capsules.

2. The implantable drug delivery device of claim 1 further comprising a drug capsule in the reservoir.

3. The implantable drug delivery device of claim 2 further comprising a carrier fluid in the reservoir, wherein the carrier fluid will dissolve the drug when the drug is released from the drug capsule, and wherein the pump is configured to convey the carrier fluid and drug capsule to the catheter.

4. The device of claim 1, wherein the drug releaser comprises an ultrasonic sound emitter that emits sufficient sound waves to release the drug from the drug capsule.

5. The device of claim 1, wherein the drug releaser from the capsule comprises a mechanical crushing or grinding device that exerts sufficient force to break the capsule open.

6. The device of claim 1, wherein the drug releaser comprises a chemical dissolving or chemical splitting apparatus to release the drug from the capsule.

7. The device of claim 6, wherein the chemical dissolving or chemical splitting apparatus is citric acid.

8. The device of claim 6, wherein the catheter further comprises a second lumen for delivering the chemical dissolving or chemical splitting apparatus to the first lumen.

9. The device of claim 8, wherein the catheter includes a port connecting the first lumen to the second lumen.

10. The device of claim 9, wherein the catheter includes a valve located at the port for controlling the movement of fluid through the port.

11. The device of claim 1, wherein the drug releaser comprises an electrical current emitter to potentiate a chemical reaction in the capsule sufficient to release the drug from the capsule.

12. The device of claim 1, wherein the drug releaser comprises a heater that conveys sufficient heat to the capsule to release the drug from the capsule.

13. The device of claim 1, wherein the drug releaser comprises a pressure device that exerts sufficient pressure on the capsule to break the capsule open to enable release of the drug from the capsule.

14. The device of claim 1, wherein the device further has a sensor that senses the physiological conditions at a target site.

15. The device of claim 1, wherein the device is programmed for drug delivery to a target site.

16. The device of claim 1, wherein the device is programmed for drug delivery to a target site via telemetry.

17. The device of claim 14, wherein the device has an electrical control circuit that receives sensed signals from the sensor and controls the drug releaser based on the sensed signals.

18. The device of claim 14, wherein the device has an electrical control circuit that receives sensed signals from the sensor and controls the amount of the drug delivered based on the sensed signals.

19. The device of claim 14, wherein the device has a drug releaser responsive to the sensed signal for regulating a therapeutic dosage of the drug to the target site.

20. The device of claim 1, wherein the device further has a premixing vessel that contains the drug capsules and that delivers the drug capsules to the reservoir for mixing with the carrier fluid.

21. The device of claim 1, further comprising a filter in the catheter, the filter located between the drug releaser and the distal end of the catheter.

22. A method for drug delivery by an implantable pump and a catheter having a discharge portion and having a proximal end coupled to said pump, said method comprising:
implanting the pump in a patient;
implanting said catheter so that said discharge portion lies adjacent a predetermined infusion site in the patient;
supplying to a reservoir at least one capsule containing a drug;
supplying to the reservoir a carrier fluid capable of dissolving the drug;
releasing the drug from the capsule in the catheter;
dissolving the drug in the carrier fluid to form a mixture; and
pumping the mixture to and through the discharge portion of the catheter and to the infusion site.

23. The method of claim 22, wherein releasing the drug from the capsule comprises subjecting the capsule to sound waves sufficient to break open the capsule.

24. The method of claim 22, wherein releasing the drug from the capsule comprises subjecting the capsule to a mechanical force sufficient to break open the capsule.

25. The method of claim 22, wherein releasing the drug from the capsule comprises subjecting the capsule to a chemical sufficient to dissolve or split the capsule.

26. The method of claim 22, wherein releasing the drug from the capsule comprises subjecting the capsule to an electrical current to potentiate a chemical reaction in the capsule sufficient to release the drug from the capsule.

27. The method of claim 22, wherein releasing the drug from the capsule comprises subjecting the capsule to heat sufficient to release the drug from the capsule.

28. The method of claim 22, wherein releasing the drug from the capsule comprises subjecting the capsule to pressure sufficient to release the drug from the capsule.

29. The method of claim 22, wherein the method further comprises sensing a physiological condition at the infusion site.

30. The method of claim 22, wherein the method further comprises sensing an amount of a substance related to a physiological condition at the infusion site.

31. The method of claim 29, wherein the method further comprises controlling the amount of drug delivery to the infusion site based on the physiological condition sensed at the infusion site.

32. The method of claim 30, wherein the method further comprises controlling the amount of drug delivery to the infusion site based on the amount of the substance sensed at the infusion site.

33. The method of claim 22, wherein the method further comprises programming an electrical circuit to control the amount of drug delivery to the infusion site.

34. The method of claim 22, wherein the method further comprises programming an electrical circuit via telemetry to control the amount of drug delivery to the infusion site.

35. The method of claim 22, wherein the method further comprises removing capsules after the drug is released from the capsule.

36. The method of claim 35, wherein the method further comprises replenishing the reservoir with at least one new capsule containing a drug through a port in the reservoir.

37. The method of claim 22, wherein the method further comprises controlling the delivery of the drug to the infusion site based on a measurement of the concentration of the drug within the carrier fluid.

38. The method of claim 22, wherein the method further comprises implanting in the patient a premixing vessel containing the capsulated drug, and supplying the capsulated drug to the reservoir from the premixing vessel.

39. The method of claim 22, further comprising filtering the carrier fluid and drug after the drug has been released from the capsule.

40. The method of claim 22, further comprising removing capsules through a catheter access port.

41. A catheter comprising:
a first lumen;
a second lumen; and
a port connecting the first lumen to the second lumen allowing mixing of two fluids within the catheter.
